(19) United States
(12) Patent Application Publication
(10) Pub. No.: US 2026/0306263 A1
(43) Pub. Date: Oct. 8, 2026

(54) MODULAR BONE DEFECT REPAIR SYSTEM

(71) Applicant: Colorado State University Research Foundation, Fort Collins, CO (US)

(72) Inventors: David Prawel, Loveland, CO (US); Vail Baumer, Fort Collins, CO (US); Tyler Gerry, Fort Collins, CO (US); Daniel Watling, Fort Collins, CO (US); Shashank Kanakamedala, Fort Collins, CO (US); Adam Schneiderhan, Fort Collins, CO (US)

(21) Appl. No.: 19/642,444

(22) Filed: Apr. 8, 2026

Related U.S. Application Data
(60) Provisional application No. 63/785,531, filed on Apr. 8, 2025.

(51) Int. Cl. A61F 2/28; A61L 27/12; A61L 27/18; A61L 27/36

(52) U.S. Cl. CPC A61F 2/28; A61L 27/12; A61L 27/18; A61L 27/365; A61F 2310/00293; A61F 2310/00359; A61L 2400/02

(57) ABSTRACT

Various implementations include a modular bone defect repair system. The system includes a plurality of scaffold segments and a sleeve having an interior cavity configured to receive the plurality of scaffold segments in an assembled configuration. Each scaffold segment has a porous architecture configured for bone tissue ingrowth. Each scaffold segment includes at least one interlocking feature including a stepped profile configured to mechanically engage with a complementary stepped profile on an adjacent scaffold segment. The sleeve constrains motion of the scaffold segments relative themselves and the host bones. Various other implementations include a scaffold having a porous architecture and a sleeve that attaches to a fixation device and surrounds at least a portion of the scaffold. The sleeve has a wall with an engineered geometry. The engineered geometry is configured to control strain within the scaffold when the device is subjected to loading.

### STATEMENT REGARDING FEDERALLY SPONSORED RESEARCH OR DEVELOPMENT

This invention was made with government support under AR080938 awarded by the National Institutes of Health. The government has certain rights in the invention.

### BACKGROUND

Bone defects resulting from trauma, disease, or surgical resection present ongoing challenges in orthopedic medicine. Critically sized bone defects, characterized by bone loss that exceeds the body's natural regenerative capacity, affect a substantial number of patients annually and are associated with considerable healthcare costs. These defects may arise from various causes including high-energy trauma, tumor resection, infection, and degenerative conditions.

Current clinical approaches for treating large bone defects include autologous bone grafting, allogeneic bone grafting, and the use of synthetic bone substitutes. Autologous bone grafts, harvested from a secondary site on the patient, remain a common treatment option but are limited by the quantity of harvestable tissue and create a secondary surgical site that may be associated with donor site morbidity. Allogeneic grafts address some limitations of autografts but introduce concerns related to disease transmission and immune response.

Synthetic scaffolds have emerged as alternatives to biological grafts for bone regeneration applications. These scaffolds are designed to provide a three-dimensional environment that supports cell attachment, proliferation, and differentiation while facilitating the transport of nutrients and waste products. Calcium phosphate-based ceramics, including hydroxyapatite and tricalcium phosphate, have been investigated for scaffold applications due to their compositional similarity to natural bone mineral and their favorable biological properties.

Mechanical stabilization of bone defect sites during the healing process presents additional considerations. Fixation devices such as plates and screws are commonly employed to maintain alignment and provide load-bearing support while bone regeneration occurs. The interaction between scaffold materials and fixation hardware, as well as the mechanical environment at the defect site, may influence healing outcomes.

The treatment of large bone defects in load-bearing applications involves balancing multiple factors including scaffold bioactivity, mechanical properties, mechanobiological considerations such as intersegmental mobility, and the protection of scaffold materials during the healing period. Ceramic scaffolds, while exhibiting favorable biological properties, may have limited mechanical strength that affects their suitability for certain applications without additional support structures.

### SUMMARY

Various implementations include a modular bone defect repair system. The system includes a plurality of scaffold segments and a sleeve having an interior cavity configured to receive the plurality of scaffold segments in an assembled configuration. Each scaffold segment has a porous architecture configured for bone tissue ingrowth. Each scaffold segment includes at least one interlocking feature including a stepped profile configured to mechanically engage with a complementary stepped profile on an adjacent scaffold segment to reduce relative micro-motion between segments. The sleeve constrains scaffold mobility in the defect site and relative motion between the assembled scaffold segments.

In some implementations, the porous architecture includes a triply periodic minimal surface structure. In some implementations, the triply periodic minimal surface structure includes a gyroid topology. In some implementations, the triply periodic minimal surface structure includes a Fischer-Koch S topology.

In some implementations, each scaffold segment includes a calcium phosphate-based ceramic material. In some implementations, the calcium phosphate-based ceramic material includes hydroxyapatite. In some implementations, the calcium phosphate-based ceramic material includes beta-tricalcium phosphate. In some implementations, the calcium phosphate-based ceramic material includes a biologically-derived bone powder.

In some implementations, each scaffold segment has a porosity in a range of 50% to 90%. In some implementations, each scaffold segment has a porosity of approximately 70%.

In some implementations, each scaffold segment includes a central canal extending therethrough configured to receive a growth factor delivery medium. In some implementations, the central canal is configured to receive bone morphogenetic protein.

In some implementations, the sleeve includes a biodegradable polymer. In some implementations, the biodegradable polymer includes polycaprolactone.

In some implementations, the sleeve includes a channel configured to receive a fixation plate. In some implementations, the channel extends along a longitudinal axis of the sleeve. In some implementations, the system further includes one or more biodegradable plugs configured to fill unused screw holes in the fixation plate. In some implementations, the biodegradable plugs are configured to prevent bone growth through the unused screw holes. In some implementations, the biodegradable plugs include polycaprolactone.

In some implementations, the sleeve includes a plurality of fenestrations configured to permit transcortical fluid flow.

In some implementations, the sleeve includes at least one fixation aperture configured to receive a bone fixation screw.

In some implementations, the stepped profile includes a stair-step configuration having a step height of at least 5 mm.

In some implementations, the plurality of scaffold segments includes at least three scaffold segments.

In some implementations, each scaffold segment includes a guide rail configured to align with a corresponding guide channel in the sleeve.

In some implementations, the sleeve has a proximal end configured to overlap with a proximal host bone and a distal end configured to contact a distal host bone. In some implementations, the sleeve has a tapered configuration along its longitudinal axis to constrain axial motion of the assembled scaffold segments.

In some implementations, the interior cavity of the sleeve is shaped to conform to a patient-specific bone geometry derived from computed tomography data. In some implementations, the interior cavity of the sleeve conforms to the patient-specific bone geometry to minimize movement of the assembled scaffold segments relative to the host bone.

In some implementations, each scaffold segment has a pore size in a range of 300 micrometers to 1200 micrometers.

Various other implementations include a bone defect repair device. The device includes a scaffold having a porous architecture and a sleeve surrounding at least a portion of the scaffold. The sleeve has a wall with an engineered geometry. The engineered geometry is configured to induce a controlled strain in the scaffold when the device is subjected to axial loading. In some implementations, the controlled strain is regulated through Poisson's effect.

In some implementations, the porous architecture includes a triply periodic minimal surface structure. In some implementations, the triply periodic minimal surface structure includes a gyroid topology. In some implementations, the triply periodic minimal surface structure includes a Fischer-Koch S topology.

In some implementations, the scaffold includes a calcium phosphate-based ceramic material. In some implementations, the calcium phosphate-based ceramic material includes hydroxyapatite. In some implementations, the calcium phosphate-based ceramic material includes beta-tricalcium phosphate. In some implementations, the calcium phosphate-based ceramic material includes a biologically-derived bone powder.

In some implementations, the sleeve includes a biodegradable polymer. In some implementations, the biodegradable polymer includes polycaprolactone.

In some implementations, the engineered geometry includes a rectilinear infill pattern.

In some implementations, the wall has a thickness configured to control micro-motion of the scaffold under physiological loading conditions. In some implementations, the thickness is in a range of 1.6 mm to 3 mm.

In some implementations, the sleeve includes a channel configured to receive a fixation plate. In some implementations, the channel extends along a longitudinal axis of the sleeve. In some implementations, the device further includes one or more biodegradable plugs configured to fill unused screw holes in the fixation plate. In some implementations, the biodegradable plugs are configured to prevent bone growth through the unused screw holes. In some implementations, the biodegradable plugs include polycaprolactone.

In some implementations, the sleeve includes a plurality of fenestrations configured to permit transcortical fluid flow. In some implementations, the plurality of fenestrations have a sinusoidal configuration.

In some implementations, the scaffold has a porosity in a range of 50% to 90%. In some implementations, the scaffold has a porosity of approximately 70%.

In some implementations, the scaffold includes a central canal extending therethrough configured to receive a growth factor delivery medium. In some implementations, the central canal is configured to receive bone morphogenetic protein.

### BRIEF DESCRIPTION OF DRAWINGS

Example features and implementations of the present disclosure are disclosed in the accompanying drawings. However, the present disclosure is not limited to the precise arrangements and instrumentalities shown. Similar elements in different implementations are designated using the same reference numerals.

FIG. 1 is an isometric view of a modular bone defect repair system, according to one implementation.

FIG. 2 is a cross-sectional view of the modular bone defect repair system shown in FIG. 1 along line 2-2.

FIG. 3 is an isometric view of a triply periodic minimal surface structure for a scaffold segment, according to one implementation.

FIG. 4 is an isometric view of scaffold segments of a modular bone defect repair system, according to one implementation.

FIG. 5A is an isometric view of scaffold segments of a modular bone defect repair system, according to another implementation.

FIG. 5B is an isometric view of the scaffold segments shown in FIG. 5A from different orientations.

FIG. 6 is an isometric view of a modular bone defect repair device, according to another implementation.

FIG. 7 is an isometric view of a sleeve of a modular bone defect repair system, according another implementation.

FIG. 8 is an isometric view of a bone defect repair system, according to another implementation.

FIG. 9 is an isometric view of a modular bone defect repair system installed on a skeletal structure, according to another implementation.

FIG. 10 is an isometric view of a bone defect repair system in a full construct configuration, according to another implementation.

FIG. 11 is an isometric view of two sleeves of a modular bone defect repair system, according to another implementation.

FIG. 12 is an isometric view of a scaffold segment of FIG. 11 and a scaffold segment of a modular bone defect repair system, according to another implementation.

### DETAILED DESCRIPTION

The following detailed description is merely exemplary in nature and is not intended to limit the disclosure or the application and uses of the disclosure. Furthermore, there is no intention to be bound by any theory presented in the preceding background or the following detailed description.

The present disclosure relates to systems and devices for treating bone defects, and more particularly to modular scaffold-based systems that provide both structural support and an environment conducive to bone regeneration. The disclosed systems address limitations of existing approaches by combining multiple scaffold segments within a protective sleeve that constrains relative motion between segments while permitting controlled mechanical stimulation of the scaffold structure.

Conventional approaches to treating large bone defects face several challenges. Autologous bone grafts are limited by the quantity of available donor tissue and create secondary surgical sites with associated morbidity. Allogeneic grafts introduce concerns regarding disease transmission and immune response. Synthetic scaffolds fabricated from calcium phosphate-based ceramics offer favorable biological properties including osteoconductivity and osteointegration, but the brittle nature of these materials limits their mechanical performance in load-bearing applications without additional support structures.

The modular bone defect repair systems disclosed herein address these challenges through several features. The use of multiple interlocking scaffold segments enables treatment of defects of varying sizes without requiring fabrication of a single monolithic scaffold structure for each defect dimension. The interlocking features between adjacent scaffold segments provide alignment and mechanical engagement that maintains scaffold integrity during assembly and implantation. The sleeve surrounding the assembled scaffold segments protects the brittle ceramic material from impact and shear forces while constraining relative motion between segments.

The sleeve configuration further provides mechanical benefits through its engineered geometry. When the device is subjected to axial loading, the sleeve wall responds through Poisson's effect to induce controlled strain in the scaffold. This controlled strain promotes mechanotransduction processes that stimulate bone cell activity and accelerate bone regeneration within the porous scaffold architecture. The sleeve also accommodates a fixation plate that provides primary load-bearing support, transferring mechanical loads around the defect site during the healing process.

This application is related to U.S. patent application Ser. No. 18/624,342, the entire contents of which are incorporated herein by reference.

Various implementations include a modular bone defect repair system. The system includes a plurality of scaffold segments and a sleeve having an interior cavity configured to receive the plurality of scaffold segments in an assembled configuration. Each scaffold segment has a porous architecture configured for bone tissue ingrowth. Each scaffold segment includes at least one interlocking feature including a stepped profile configured to mechanically engage with a complementary stepped profile on an adjacent scaffold segment. The sleeve constrains relative motion between the assembled scaffold segments.

Various other implementations include a bone defect repair device. The device includes a scaffold having a porous architecture and a sleeve surrounding at least a portion of the scaffold. The sleeve has a wall with an engineered geometry. The engineered geometry is configured to induce a controlled strain in the scaffold when the device is subjected to axial loading. The controlled strain is regulated through Poisson's effect.

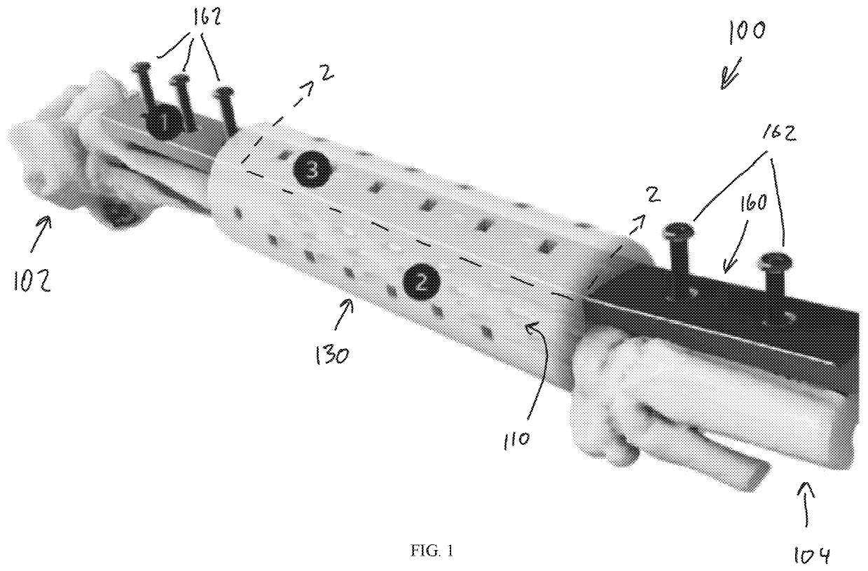

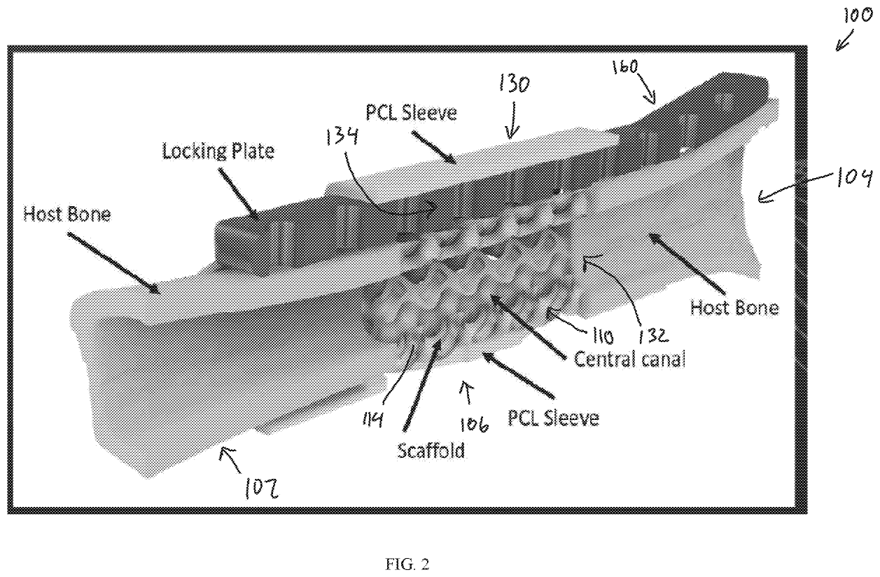

Referring to FIG. 1 and FIG. 2, a modular bone defect repair system 100 is configured to treat bone defects by bridging a gap between proximal and distal host bone segments 102, 104. The modular bone defect repair system 100 includes three primary components that work together to provide both mechanical stability and an environment conducive to bone regeneration: a plurality of scaffold segments 110, a sleeve 130, and a fixation plate 160.

The plurality of scaffold segments 110 are configured to fill the void 106 created by a bone defect. Each scaffold segment has a porous architecture 114 configured for bone tissue ingrowth. The porous architecture 114 provides a three-dimensional environment that mimics properties of trabecular bone, facilitating cellular migration, nutrient transport, and waste removal during the bone regeneration process. The scaffold segments 110 may be fabricated from calcium phosphate-based ceramic materials, which exhibit high levels of bioactivity including osteoconductivity, osteoinductivity, and osteointegration. The scaffold segments 110 may exhibit compressive strength in a range of 0.63-1.83 MPa, which matches the low end of human trabecular bone.

As shown in FIG. 2, a sleeve 130 has an interior cavity 132 configured to receive the plurality of scaffold segments 110 in an assembled configuration. The sleeve 130 surrounds the assembled scaffold segments 110 and constrains relative motion between the assembled scaffold segments 110. The sleeve 130 provides mechanical protection to the scaffold segments 110, which may be fabricated from brittle ceramic materials that exhibit favorable osteogenic properties but limited resistance to impact and shear forces. By constraining the scaffold segments 110 within the interior cavity 132, the sleeve 130 immobilizes the scaffold segments 110 relative to one another and relative to the surrounding bone tissue, enhancing the rate of new bone growth and reducing the risk of scaffold fracture during the healing process.

With continued reference to FIG. 1, a fixation plate 160 extends along one side of the assembly, spanning from the proximal host bone segment 102 to the distal host bone segment 104. The fixation plate 160 provides primary load-bearing support for the construct, transferring mechanical loads around the defect site during the healing process. Fixation screws 162 secure the fixation plate 160 to both the proximal and distal host bone segments 102, 104, anchoring the entire assembly in position. The sleeve 130 includes a channel 134 that accommodates the fixation plate 160, allowing the plate to pass through while maintaining contact with the scaffold segments 110.

The modular scaffold approach enables treatment of defects of varying sizes. The system 100 may be configured for defects of any length. In some implementations, the system is configured for defects ranging from 11 to 16 cm in length, which represent super-critical defects that may involve removal of approximately 40% of the bone length. The scaffold segments 110 may be designed to replace the distal approximately 40% of the length of a radius bone. By using multiple scaffold segments 110 that can be assembled within the sleeve 130, the system 100 accommodates defects of different dimensions without requiring fabrication of a single monolithic scaffold structure for each defect size.

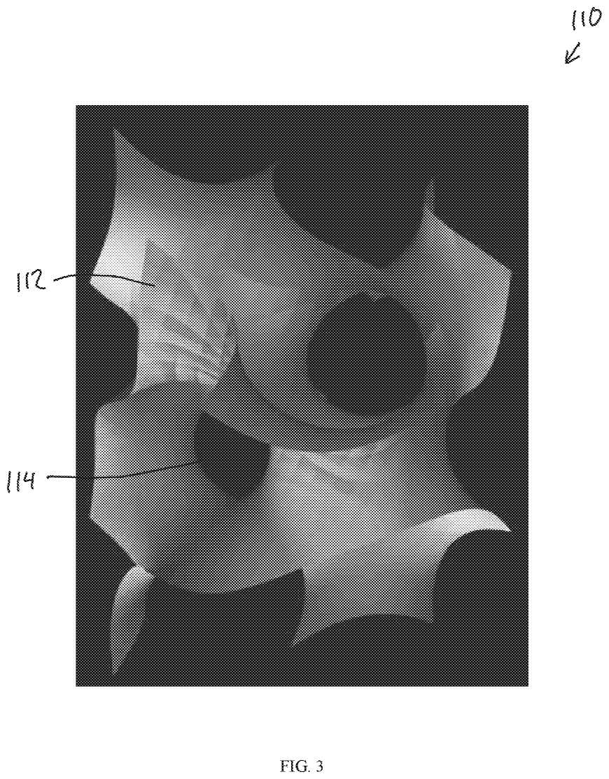

Referring to FIG. 3, a scaffold segment 110 has a porous architecture 114 comprising a triply periodic minimal surface structure. The triply periodic minimal surface structure shown in FIG. 3 comprises a Fischer-Koch S topology. The Fischer-Koch S topology exhibits a continuous, interconnected network of curved surfaces that form a repeating pattern in three dimensions. The structure features smooth, flowing surfaces with no sharp corners or junctions, which are characteristic of triply periodic minimal surfaces. The curved walls 112 of the scaffold wind through space, creating a network of passages that extend in multiple directions. The interconnected pore structure visible throughout the scaffold provides pathways for fluid flow and cellular migration.

The porous architecture 114 of the scaffold segment comprises an interconnected pore network that facilitates bone tissue ingrowth, nutrient transport, and waste removal during bone regeneration. The triply periodic minimal surface structure creates a continuous interior devoid of sharp corners and junctions within each unit cell. The smooth internal surfaces provide high surface area per volume, which promotes cell attachment, proliferation, and differentiation. The interconnected channels permit mass transport of nutrients, gases, and waste products throughout the scaffold volume.

As shown in FIG. 3, the scaffold segment, in some implementations, can have a porosity in a range of 50% to 90%. In some implementations, the scaffold segment has a porosity of approximately 70%, which falls within the middle of the porosity range used for bone tissue engineering applications. The porosity range mimics natural trabecular bone, which has a honeycomb-like internal structure with porosity varying depending on anatomical location. In some implementations, the scaffold segment has a pore size in a range of 300 micrometers to 1200 micrometers. In some implementations, the scaffold segment has a pore size in a range of 585 micrometers to 723 micrometers as measured by micro-computed tomography. In some implementations, the scaffold segment has a wall thickness of approximately 0.42-0.43 mm as measured by micro-computed tomography. In some implementations, the scaffold segment has a specific surface area of approximately 5.5-5.8 mm⁻¹ as measured by micro-computed tomography.

In some implementations, the scaffold segment comprises a calcium phosphate-based ceramic material. In some implementations, the calcium phosphate-based ceramic material comprises hydroxyapatite. In some implementations, the hydroxyapatite particles have a needle-like morphology with particle size of approximately 85-89 nm. In some implementations, the scaffold segment has a permeability in a range of 1.0×10⁻⁹ to 2.5×10⁻⁹ m², which is comparable to trabecular bone.

The scaffold segment 110 can be fabricated using a photocasting process that combines viscous extrusion of a ceramic slurry with simultaneous layer-wise photopolymerization using UV light at 405 nm wavelength. In some implementations, the ceramic slurry comprises ethylene glycol dimethacrylate (EGDMA) as a monomer, diphenyl(2,4,6-trimethylbenzoyl)phosphine oxide (TPO) as a photoinitiator, and Lubrizol Solsperse AC7550 as a dispersant. In some implementations, the ceramic slurry comprises Solplus D540 as a dispersant. In some implementations, the ceramic slurry has a solid loading of approximately 41 volume percent hydroxyapatite particles. In some implementations, the ceramic slurry is prepared using a planetary ball mill with agate milling media at speeds of 120-360 rpm depending on process cycle.

In some implementations, the scaffold segment 110 is fabricated using a Hyrel 3D printer with an EMO-XT viscous extrusion print head equipped with a 405 nm LED array providing light power density of 0.7-1.3 mW/cm². In some implementations, the scaffold segment 110 is printed using a 22-gauge (0.413 mm) dispensing tip, which determines the wall thickness of the scaffold struts. In some implementations, the scaffold segment 110 has a layer height of 0.2 mm during the 3D printing process. The scaffold segment 110 can be printed onto a bed covered with painter's tape to improve bed adhesion and prevent reflected light from prematurely curing slurry in the nozzle. In some implementations, the scaffold segment 110 is post-cured under LED for an additional 3 minutes after printing before sintering.

In some implementations, the scaffold segment 110 is sintered at 1200 °C for 3 hours with a ramp rate of 5 °C/min to remove organic content and densify the ceramic. In some implementations, the scaffold segment 110 can experience approximately 21-25% shrinkage during the sintering process, which is accounted for by scaling up the as-designed dimensions.

The scaffold architecture can be generated using software that creates 3D printable models from trigonometric approximations of triply periodic minimal surface functions using a marching cubes iso-contouring algorithm. The scaffold wall thickness can be created using a gradient offset method that adds the magnitude of the gradient vector to the original implicit function for uniform offset distance.

In some implementations, such as the scaffold segment shown in FIG. 2, the triply periodic minimal surface structure comprises a gyroid topology. The gyroid topology provides an alternative porous architecture 114 with interconnected channels suitable for bone tissue ingrowth. The gyroid topology has many of the same features and benefits as the Fischer-Koch S topology shown in FIG. 3. In some implementations, the calcium phosphate-based ceramic material comprises beta-tricalcium phosphate. Beta-tricalcium phosphate exhibits higher degradation rates compared to hydroxyapatite, which may be desirable for certain bone regeneration applications. In some implementations, the calcium phosphate-based ceramic material comprises a biologically-derived bone powder.

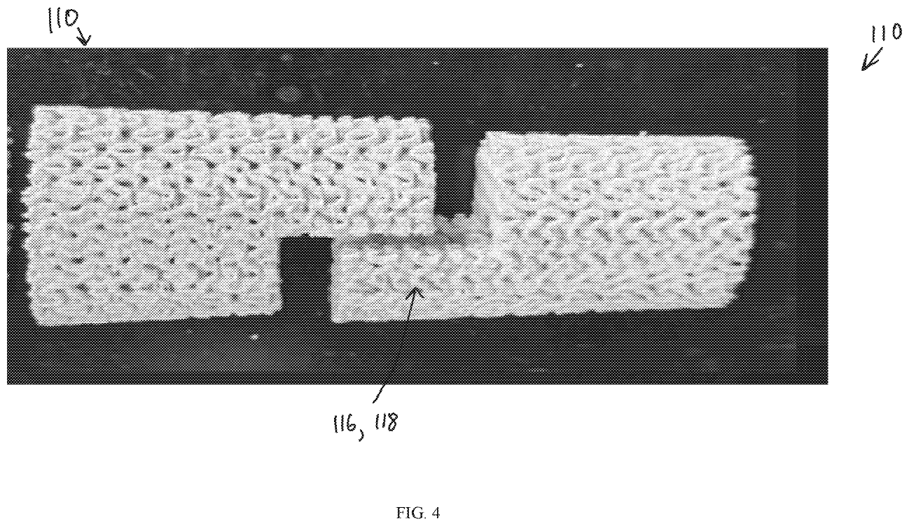

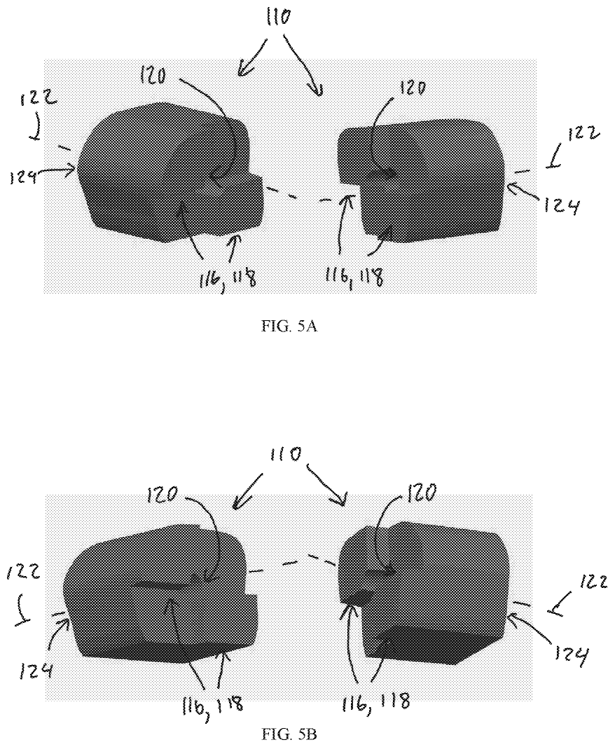

Referring to FIGS. 4, 5A, and 5B, each scaffold segment 110 includes at least one interlocking feature 116 comprising a stepped profile 118 configured to mechanically engage with a complementary stepped profile 118 on an adjacent scaffold segment 110. The stepped profile 118 comprises a stair-step configuration that enables mechanical engagement between adjacent scaffold segments 110 when assembled within the sleeve 130. As shown in FIGS. 4, 5A, and 5B, a first scaffold segment 110 includes a protruding stepped portion that extends from a mating surface of the first scaffold segment 110, while a second scaffold segment 110 includes a corresponding recessed stepped portion configured to receive the protruding stepped portion of the first scaffold segment 110. The complementary protruding and recessed stepped portions on adjacent scaffold segments 110 provide alignment and stability within the sleeve assembly.

In some implementations, the stepped profile 118 comprises a stair-step configuration having a step height of at least 5 mm. In some implementations, the stair-step configuration has a step height of at least 10 mm between adjacent scaffold segments 110. The step height dimensions are sized to maintain structural integrity given the brittle nature of the calcium phosphate-based ceramic material from which the scaffold segments 110 are fabricated. The interlocking features 116 provide sufficient mechanical engagement between adjacent scaffold segments 110 while allowing for assembly within the constraining sleeve 130.

In some implementations, the plurality of scaffold segments 110 comprises at least three scaffold segments 110. In some implementations, four scaffold segments 110 are assembled to fill a full defect region. By dividing the scaffold into multiple segments rather than fabricating a single monolithic structure, the system 100 accommodates the fabrication constraints associated with photocasting ceramic materials while enabling treatment of defects of varying sizes including very large defects.

In some implementations, each scaffold segment has a length in a range of 3 cm to 4 cm. In some implementations, scaffold segments have lengths shorter or longer than this range depending on the defect geometry. In some implementations, the system includes a kit comprising a plurality of scaffold segments having different lengths, enabling selection of scaffold segments during the surgical procedure to achieve a desired total scaffold length that matches the defect dimension. In some implementations, some scaffold segments include interlocking features on both ends while other scaffold segments include interlocking features on only one end or no interlocking features, depending on the position of the scaffold segment within the assembled configuration.

In some implementations, the interlocking features 116 have alternative configurations that provide mechanical engagement between adjacent scaffold segments 110. In some implementations, the step height varies along the width of the stepped profile 118 to accommodate variations in scaffold segment geometry.

Each scaffold segment 110 includes a central canal 120 extending therethrough configured to enhance intramedullary fluid flow or to receive a growth factor delivery medium. The central canal 120 extends along a longitudinal axis 122 of each scaffold segment 110, creating a continuous pathway through the assembled scaffold structure when multiple scaffold segments 110 are positioned in series within the sleeve 130. In some implementations, the central canal 120 has a cross-sectional dimension of approximately 6 mm×6 mm, which accommodates insertion of growth factor delivery media while maintaining sufficient wall thickness in the surrounding scaffold structure for mechanical integrity.

The growth factor delivery medium comprises an absorbable media such as a collagen sponge 124. In some implementations, the collagen sponge 124 is cut into pieces approximately 1 inch×1 inch×2 inches prior to loading into the central canal 120. The collagen sponge 124 pieces can be threaded through the central canal 120 using suture material. A threading technique can involve tying one end of a suture thread to a piece of collagen sponge 124 and threading the suture through the central canal 120 of the scaffold segment 110. The suture can be pulled to draw the collagen sponge 124 through the central canal 120 until the collagen sponge 124 is flush with a far end of the scaffold segment 110. A second collagen sponge 124 piece can then be loaded using a longer suture thread, with the second collagen sponge 124 entering the central canal 120 and meeting the first collagen sponge 124. The suture threads can be cut away after loading, along with any collagen sponge 124 material that protrudes from the ends of the scaffold segment 110 and could prevent adjacent scaffold segments 110 from mating properly. At a proximal scaffold segment 110 where the scaffold meets host bone, the collagen sponge 124 can be configured to protrude from the end of the scaffold segment 110 and extend into a medullary cavity of the host bone.

In some implementations, the central canal 120 is configured to receive bone morphogenetic protein. In some implementations, the bone morphogenetic protein is recombinant human BMP-2 (rhBMP-2). In some implementations, the rhBMP-2 is delivered at a concentration of approximately 1.43 mg/mL with a total dose of approximately 2 mg per patient. In some implementations, the rhBMP-2 is loaded into the collagen sponge 124 after the collagen sponge 124 has been threaded through the central canal 120. A syringe loaded with the rhBMP-2 solution can be inserted through the central canal 120, and the syringe can be drawn out while dispensing an even amount of rhBMP-2 over the length of the collagen sponge 124. An even fraction of the total volume can be dispensed into each scaffold segment 110, with one-quarter of the volume dispensed into each of four scaffold segments 110 or one-third of the volume dispensed into each of three scaffold segments 110.

In some implementations, the system 100 includes antibiotic delivery via collagen sponges loaded with amikacin. In some implementations, the amikacin is applied at a dose of 25 mg/kg. The antibiotic-loaded collagen sponges can be applied in an area surrounding the sleeve 130 rather than within the central canal 120 of the scaffold segments 110.

In some implementations, the central canal 120 is configured to receive growth factor delivery media other than collagen sponges. In some implementations, the bone morphogenetic protein comprises BMP-7 rather than rhBMP-2. In some implementations, the growth factor delivery medium comprises a polymer carrier configured for sustained release of bone morphogenetic protein. In some implementations, the central canal 120 has cross-sectional dimensions other than 6 mm×6 mm to accommodate different growth factor delivery media or anatomical requirements. In some implementations, the antibiotic comprises agents other than amikacin for infection prevention at the surgical site.

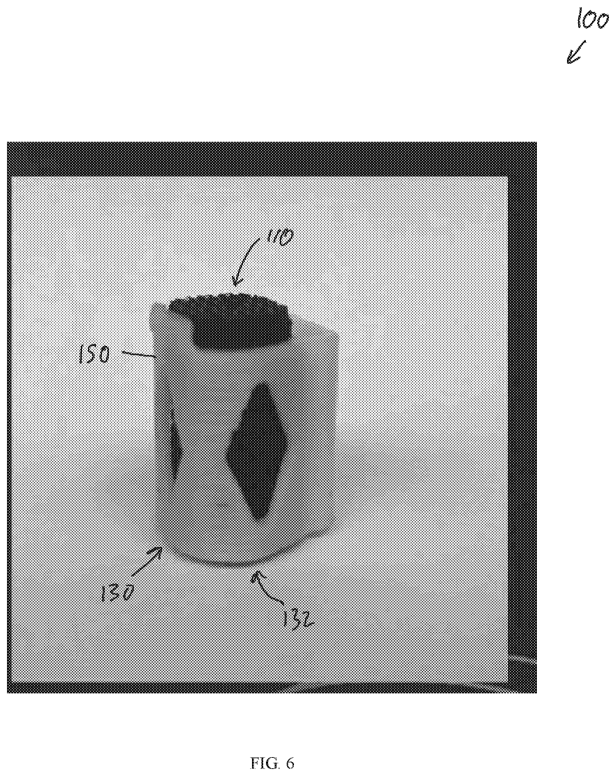

Referring to FIG. 6, the sleeve 130 comprises a biodegradable polymer. In some implementations, the biodegradable polymer comprises polycaprolactone. In some implementations, the polycaprolactone used for the sleeve 130 has a molecular weight of approximately 80 kDa, which provides a degradation timeline of approximately 2 years in vivo. The sleeve 130 shown in FIG. 6 surrounds at least a portion of the scaffold, with the sleeve 130 having a wall 150 with an engineered geometry. The sleeve 130 has an interior cavity 132 shaped to receive the assembled scaffold segments 110. The interior cavity 132 constrains relative motion between the assembled scaffold segments 110, protects the brittle ceramic scaffold from impact and shear forces, and directs nutrient flow through the porous scaffold structure.

In some implementations, such as the implementation shown in FIG. 6, the sleeve 130 is fabricated using melt extrusion 3D printing of polycaprolactone filament at a nozzle temperature of 120 °C and a bed temperature of 35 °C. In some implementations, the sleeve 130 is printed at 100% infill density with a layer height of 0.2 mm and a nozzle and road width of 0.4 mm. A 100% infill density provides a solid wall structure that protects the ceramic scaffold segments 110 while maintaining the engineered geometry of the sleeve wall 150. In some implementations, the sleeve 130 is designed with a minimum thickness of 2 mm at the thinnest part to maintain structural integrity during handling, implantation, and the healing process.

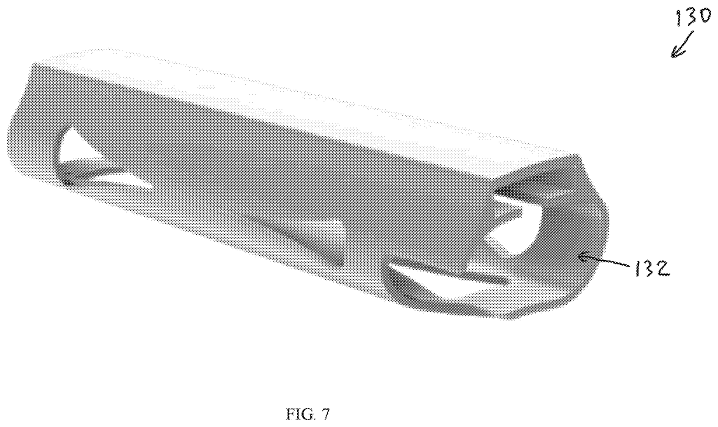

Referring to FIG. 7, another implementation of a sleeve 130 is shown with an interior cavity 132 configured to receive scaffold segments having a porous architecture. The sleeve 130 shown in FIG. 7 includes a tolerance of 10% XY added to the area surrounding the radius and 7.5% tolerance added to the area surrounding the scaffold. The tolerances accommodate variations in bone geometry and scaffold dimensions while maintaining sufficient constraint to immobilize the scaffold segments within the sleeve 130.

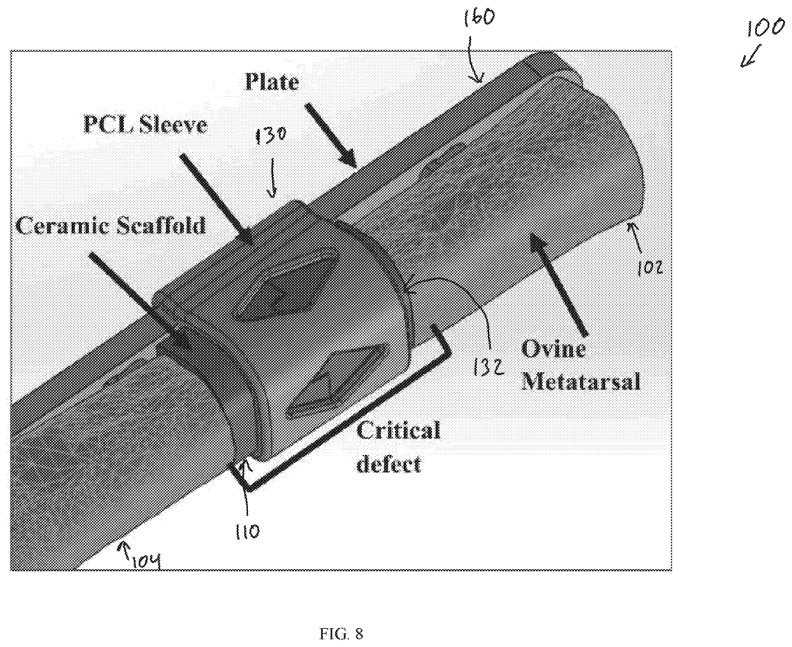

As shown in FIG. 8, another implementation of a sleeve 130 is shown that is designed as a patient-specific component derived from computed tomography data. The interior cavity 132 of the sleeve 130 is shaped to conform to a patient-specific bone geometry derived from the computed tomography data. The patient-specific design enables the sleeve 130 to interface with the proximal and distal host bone segments 102, 104 while accommodating the assembled scaffold segments 110 and the fixation plate 160. The sleeve 130 extends to overlap with the proximal host bone segment 102, providing additional stability at the bone-sleeve interface.

In some implementations, the biodegradable polymer comprises a composite including calcium phosphate and polycaprolactone. In some implementations, the sleeve 130 is fabricated at nozzle temperatures other than 120 °C to accommodate different polycaprolactone formulations or composite materials. In some implementations, the sleeve 130 is printed at infill densities less than 100% to reduce material usage or modify mechanical properties. In some implementations, the minimum wall thickness is greater than 2 mm to provide additional mechanical protection for the scaffold segments 110. In some implementations, the tolerances surrounding the radius and scaffold are adjusted to accommodate different anatomical requirements or manufacturing variations.

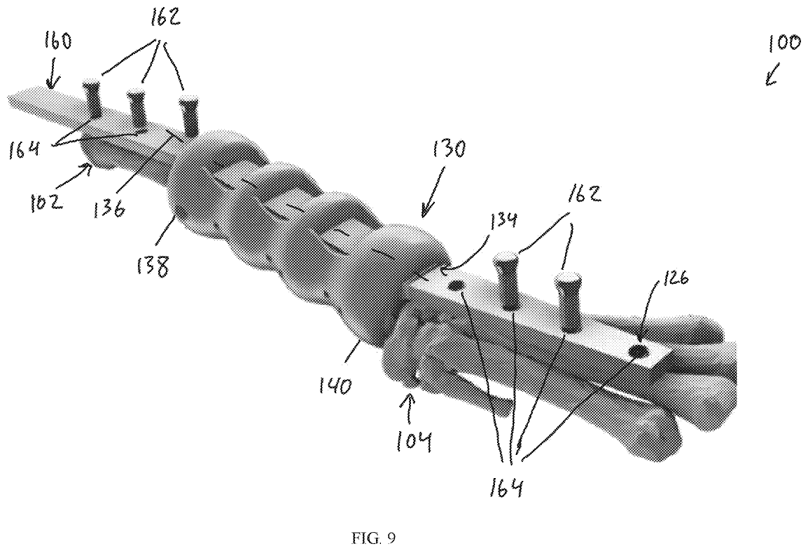

Referring to FIG. 9, the sleeve 130 includes a channel 134 configured to receive a fixation plate 160. The channel 134 extends along a longitudinal axis 136 of the sleeve 130, spanning from a proximal end 138 of the sleeve 130 to a distal end 140 of the sleeve 130. The channel 134 is configured to receive a fixation plate 160 such as a locking compression plate that provides primary mechanical stabilization to the bone defect repair construct. The fixation plate 160 is secured to both the proximal and distal host bone segments 102, 104 using fixation screws 162 that extend through screw holes 164 in the fixation plate 160 and into the host bone tissue.

The channel 134 in the sleeve 130 shown in FIG. 9 is sized up uniformly by approximately 1 mm relative to the dimensions of the fixation plate 160 to ensure proper plate fit within the channel 134. The 1 mm tolerance accommodates manufacturing variations in both the sleeve 130 and the fixation plate 160 while allowing for light press fit during assembly. The channel 134 extends along the longitudinal axis 136 of the sleeve 130, enabling the fixation plate 160 to span the entire length of the defect region while remaining seated within the sleeve structure.

The system 100 further includes one or more biodegradable plugs 126 configured to fill unused screw holes 164 in the fixation plate 160. The biodegradable plugs 126 are configured to prevent bone growth through the unused screw holes 164. The biodegradable plugs 126 comprise polycaprolactone. The biodegradable plugs 126 are inserted into the screw holes 164 in the fixation plate 160 that overlie the scaffold region, where fixation screws 162 are not used to secure the plate to bone. By filling the unused screw holes 164, the biodegradable plugs 126 prevent bone tissue from growing through the screw holes 164 during the healing process. The prevention of bone growth through the unused screw holes 164 facilitates later removal of the fixation plate 160 after the bone defect has healed, as the plate can be removed without requiring resection of bone tissue that has grown through the screw holes 164.

In some implementations, the biodegradable plugs 126 are fabricated from polycaprolactone having a molecular weight of approximately 80 kDa, matching the molecular weight of the polycaprolactone used for the sleeve 130. The matching molecular weight provides similar degradation timelines for the plugs 126 and the sleeve 130, ensuring that the plugs 126 remain in place throughout the healing period while degrading at a rate compatible with the overall construct.

In some implementations, the biodegradable plugs 126 comprise materials other than polycaprolactone, such as poly(lactic-co-glycolic acid) or other biodegradable polymers with appropriate degradation timelines. In some implementations, the biodegradable plugs 126 have different molecular weights than the sleeve 130 to achieve different degradation rates. In some implementations, the biodegradable plugs 126 are configured with interference fit dimensions that provide secure retention within the screw holes 164 without requiring adhesive bonding.

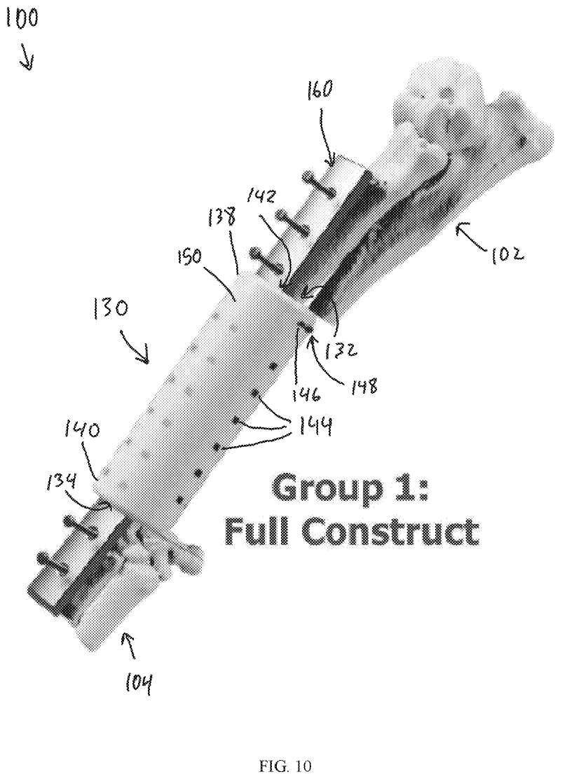

Referring to FIG. 10, the sleeve 130 includes a plurality of fenestrations 144 configured to permit transcortical fluid flow. The fenestrations 144 extend through the wall 150 of the sleeve 130, creating openings that allow fluid communication between the interior cavity 132 containing the scaffold segments 110 and the surrounding tissue environment. The fenestrations 144 permit nutrient-rich fluid to flow into the scaffold region while allowing waste products to exit, supporting the metabolic needs of cells within the porous scaffold architecture during bone regeneration.

With continued reference to FIG. 10, the plurality of fenestrations 144 have a sinusoidal configuration. The sinusoidal configuration provides elongated openings that extend along the sleeve wall 150 in a wave-like pattern. The sinusoidal fenestrations 144 provide increased open area for fluid flow compared to circular fenestrations of equivalent width while maintaining structural continuity of the sleeve wall 150 between adjacent fenestrations 144.

In some implementations, the plurality of fenestrations provide a sleeve wall porosity in a range of 10% to 50%. In some implementations, the plurality of fenestrations provide a sleeve wall porosity in a range of 20% to 40%. In some implementations, the plurality of fenestrations provide a sleeve wall porosity of approximately 27%.

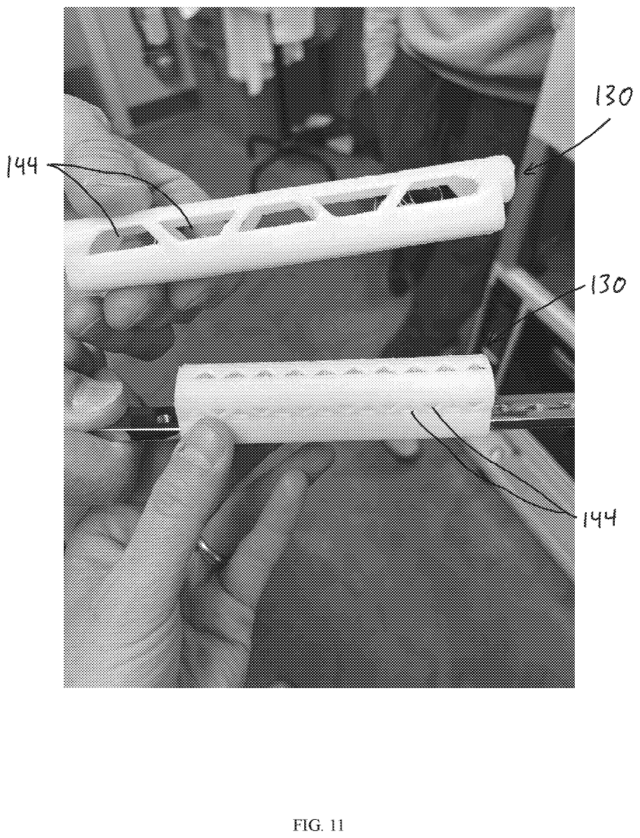

Referring to FIG. 11, two sleeves 130 are shown having different fenestration configurations. One sleeve has sinusoidal fenestrations as described above, while another sleeve has triangular fenestrations. In some implementations, the fenestrations 144 have configurations other than sinusoidal, including circular, elliptical, rectangular, or diamond-shaped openings. In some implementations, the fenestrations 144 have a triangular configuration with sides oriented at approximately 45 degrees relative to the longitudinal axis of the sleeve 130. The 45-degree orientation enables the sleeve 130 to be fabricated using melt extrusion 3D printing without requiring support structures or post-processing to remove support material from the fenestration openings. In some implementations, the fenestrations 144 are distributed in patterns other than uniform arrays, with fenestration density varying along the length of the sleeve 130 to accommodate different fluid flow requirements at different locations within the defect region. In some implementations, the fenestrations 144 are sized to permit vascular ingrowth from surrounding soft tissue into the scaffold region.

As shown in FIG. 10, the sleeve 130 includes at least one fixation aperture 146 configured to receive a bone fixation screw 148. The fixation apertures 146 are positioned at the proximal end 138 of the sleeve 130, where the sleeve 130 overlaps with the proximal host bone segment 102. Bone fixation screws 148 extend through the fixation apertures 146 and engage with the cortical bone of the proximal host bone segment 102, anchoring the sleeve 130 to the native bone tissue. The fixation apertures 146 provide additional mechanical stabilization of the sleeve 130 beyond the constraint provided by the fixation plate 160 seated within the channel 134.

In some implementations, the fixation apertures 146 are positioned at locations other than the proximal end 138 of the sleeve 130, including at the distal end 140 or at intermediate locations along the sleeve length. In some implementations, the fixation apertures 146 are configured to receive locking screws that thread into the aperture as well as into the bone, providing fixed-angle stability. In some implementations, the number of fixation apertures 146 varies based on the mechanical requirements of the specific application and the quality of the host bone tissue.

Each scaffold segment 110 includes a guide rail 142 configured to align with a corresponding guide channel in the sleeve 130. The guide rail 142 extends along the exterior surface of each scaffold segment 110, providing a protruding feature that engages with a complementary recessed channel in the interior cavity 132 of the sleeve 130. The guide rail 142 and guide channel interface constrains rotational movement of the scaffold segments 110 within the sleeve 130, maintaining proper alignment of the scaffold segments 110 relative to one another and relative to the fixation plate 160.

In some implementations, the guide rail 142 has dimensions of approximately 5 mm wide by 1 mm tall. The guide rail dimensions are sized to provide sufficient engagement with the guide channel to constrain rotational movement while maintaining structural integrity of the ceramic scaffold material. The guide rail 142 extends along the longitudinal axis 122 of each scaffold segment 110, providing continuous alignment constraint along the length of the scaffold segment 110.

In some implementations, the guide rail 142 is positioned on the scaffold segment 110 surface opposite the fixation plate channel 134, providing rotational constraint about an axis perpendicular to the plate. In some implementations, multiple guide rails 142 are positioned at different locations around the circumference of each scaffold segment 110 to provide enhanced rotational constraint. In some implementations, the guide rail 142 has a tapered cross-section that facilitates insertion of the scaffold segment 110 into the sleeve 130 while providing secure engagement once fully inserted.

The sleeve 130 has a proximal end 138 configured to overlap with a proximal host bone segment 102 and a distal end 140 configured to contact a distal host bone segment 104. The proximal end 138 of the sleeve 130 extends over a portion of the proximal host bone segment 102, creating an overlap region where the sleeve 130 surrounds both the scaffold segments 110 and a portion of the native bone. The overlap at the proximal end 138 provides additional stability at the bone-sleeve interface and constrains relative motion between the sleeve 130 and the proximal host bone segment 102.

In some implementations, the proximal overlap extends approximately 20% of the original defect length. The 20% overlap provides sufficient contact area between the sleeve 130 and the proximal host bone segment 102 to constrain relative motion while minimizing the extent of native bone that must be covered by the sleeve 130. The overlap region accommodates the fixation apertures 146 through which bone fixation screws 148 anchor the sleeve 130 to the proximal host bone segment 102.

The distal end 140 of the sleeve 130 is configured to contact the distal host bone segment 104 without substantial overlap. The distal end 140 terminates at or near the interface between the scaffold segments 110 and the distal host bone segment 104, allowing direct contact between the scaffold and the native bone tissue at the distal interface. The configuration at the distal end 140 facilitates bone tissue ingrowth from the distal host bone segment 104 into the porous scaffold architecture 114.

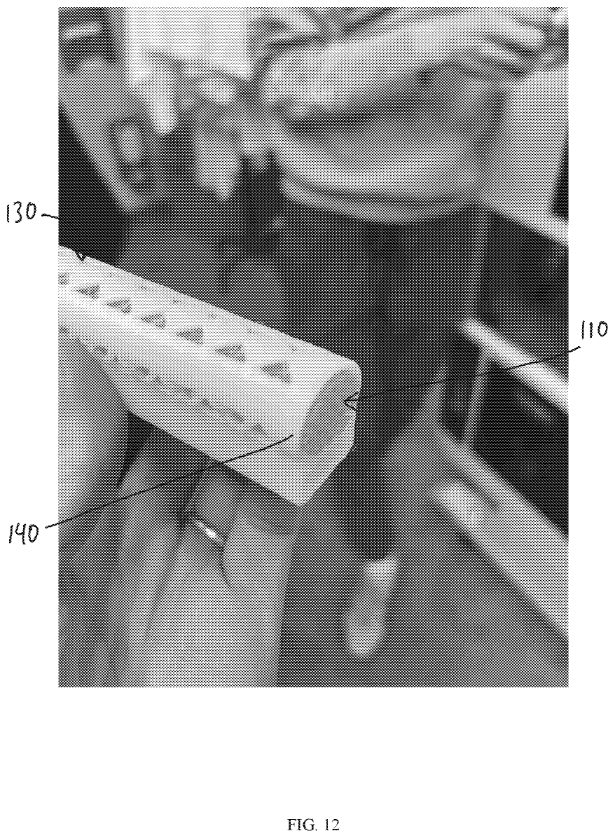

In some implementations, such as the implementation shown in FIG. 12, the scaffold segments 110 can extend beyond the distal end 140 of the sleeve 130 to directly contact the distal host bone segment 104. In some implementations, the scaffold overhang is approximately 1 mm. This configuration enables direct bone-to-scaffold contact for bone tissue integration at the distal interface. The sleeve 130 does not require direct contact with the host bone, as the fixation plate 160 provides sufficient mechanical stabilization of the construct.

In some implementations, the sleeve 130 has a tapered configuration along its longitudinal axis 136 to constrain axial motion of the assembled scaffold segments 110. The tapered configuration provides a gradually decreasing interior cavity 132 dimension from the proximal end 138 toward the distal end 140 of the sleeve 130. The taper creates a wedging effect that resists axial displacement of the scaffold segments 110 within the sleeve 130, maintaining the assembled configuration of the scaffold segments 110 during implantation and throughout the healing process.

The interior cavity 132 of the sleeve 130 is shaped to conform to a patient-specific bone geometry derived from computed tomography data. The patient-specific shaping of the interior cavity 132 enables the sleeve 130 to interface with the proximal and distal host bone segments 102, 104 while accommodating the assembled scaffold segments 110 and the fixation plate 160. The interior cavity 132 geometry is derived from three-dimensional imaging data obtained from the patient prior to surgery, enabling fabrication of a sleeve 130 that conforms to the individual patient's anatomy.

In some implementations, the interior cavity 132 of the sleeve 130 conforms to the patient-specific bone geometry to minimize rotational movement of the assembled scaffold segments 110 relative to the host bone. The conforming geometry creates an interface between the sleeve 130 and the host bone that resists torsional loads applied to the repair construct. The patient-specific shaping accounts for the natural curvature and cross-sectional variation of the bone along its length, providing a close fit that constrains relative motion between the sleeve 130 and the surrounding bone tissue.

The sleeve 130 is designed using Boolean operations that subtract the volumes of the scaffold segments 110, the fixation plate 160, and the host bone from a base sleeve geometry. The Boolean subtraction operations create the interior cavity 132 with the appropriate shape to receive each component. Tolerances are added to each subtracted volume to accommodate manufacturing variations and enable assembly of the components. In some implementations, a tolerance of 10% XY is added to the area surrounding the radius bone, and a tolerance of 7.5% is added to the area surrounding the scaffold segments 110.

In some implementations, the sleeve 130 is designed with a minimum wall thickness of 2 mm at the thinnest part to maintain structural integrity. The minimum wall thickness ensures that the sleeve 130 can withstand handling forces during implantation and physiological loads during the healing process without fracturing or deforming excessively. In some implementations, the loading conditions may include walking, trotting, running, etc. In some implementations, the loading conditions may be large on a human scale. The wall thickness varies around the circumference of the sleeve 130 based on the patient-specific geometry, with thicker regions where the bone geometry creates larger offsets from the base sleeve geometry and thinner regions where the bone geometry approaches the outer surface of the sleeve 130. The minimum wall thickness constraint is applied during the design process to ensure that no region of the sleeve wall 150 falls below the 2 mm threshold while maintaining the overall structural integrity and constraint function of the sleeve 130.

In some implementations, the proximal overlap with the host bone is greater than or less than 20% of the original defect length to accommodate different anatomical requirements or surgical approaches. In some implementations, the interior cavity 132 is shaped using imaging modalities other than computed tomography, such as magnetic resonance imaging, to derive the patient-specific bone geometry. In some implementations, the tolerances at the bone interface, scaffold interface, and plate interface are adjusted to accommodate different manufacturing processes or material properties. In some implementations, the Boolean operations incorporate additional components such as guide rails 142 or alignment features that further constrain rotational movement of the scaffold segments 110 within the sleeve 130.

The fixation plate 160 comprises a locking compression plate that extends from the proximal host bone segment 102 to the distal host bone segment 104. The locking compression plate spans the entire length of the bone defect repair construct, providing continuous mechanical support across the defect region. The fixation plate 160 is positioned within the channel 134 of the sleeve 130, with the plate seated against the scaffold segments 110 contained within the interior cavity 132 of the sleeve 130.

Fixation screws 162 secure the fixation plate 160 to both the proximal and distal host bone segments 102, 104. The fixation screws 162 extend through screw holes 164 in the fixation plate 160 and engage with the cortical bone of the host bone segments. The screws at the proximal end of the fixation plate 160 anchor the plate to the proximal host bone segment 102, while the screws at the distal end anchor the plate to the distal host bone segment 104. The fixation screws 162 provide rigid attachment between the fixation plate 160 and the native bone tissue, maintaining the position of the entire bone defect repair construct throughout the healing process.

The fixation plate 160 functions as the primary load-bearing element that transfers mechanical load around the defect site. During weight-bearing activities, axial compressive forces applied to the bone are transmitted through the fixation plate 160 rather than through the scaffold segments 110. The load transfer pathway through the fixation plate 160 bypasses the defect region, protecting the brittle ceramic scaffold segments 110 from excessive compressive forces that could cause catastrophic failure. The fixation plate 160 maintains structural continuity of the limb while the bone regeneration process occurs within the porous scaffold architecture 114.

The sleeve 130 slides onto the fixation plate 160 during assembly of the bone defect repair construct. The channel 134 in the sleeve 130 is configured to receive the fixation plate 160, with the sleeve 130 positioned over the plate after the plate has been secured to the proximal host bone segment 102. The sleeve 130 is advanced distally along the fixation plate 160 until the sleeve 130 reaches the intended position spanning the defect region. The sliding assembly enables the sleeve 130 and contained scaffold segments 110 to be positioned relative to the fixation plate 160 without requiring disassembly of the plate from the proximal host bone.

In some implementations, the scaffold exhibits elastic modulus in a range of 40-72 MPa under compression testing. The elastic modulus range reflects the mechanical stiffness of the porous scaffold architecture 114 under compressive loading conditions. The elastic modulus values fall within a range that permits controlled deformation of the scaffold under physiological loading while maintaining structural integrity sufficient to support bone tissue ingrowth.

The scaffold exhibits self-reinforcing behavior after initial failure during compressive testing. When the scaffold experiences compressive loads exceeding the initial failure threshold, fractured sections of the scaffold become lodged against intact sections of the scaffold structure. The lodging of fractured sections against intact sections creates additional load-bearing pathways within the scaffold, enabling the scaffold to continue supporting compressive loads after initial failure has occurred. The self-reinforcing behavior provides a degree of damage tolerance that reduces the risk of complete scaffold collapse under loading conditions that exceed the initial failure strength.

In some implementations, the fixation plate 160 comprises a non-locking compression plate rather than a locking compression plate. In some implementations, the fixation screws 162 comprise cortical screws, cancellous screws, or locking screws depending on the bone quality and anatomical location of the defect. In some implementations, the fixation plate 160 is pre-contoured to match patient-specific bone geometry prior to installation. In some implementations, the fixation plate 160 is bent intraoperatively to conform to the anatomy at the distal end of the construct. In some implementations, the number of fixation screws 162 at the proximal and distal ends varies based on the length of the fixation plate 160 and the quality of the host bone tissue. In some implementations, the elastic modulus of the scaffold is outside the range of 40-72 MPa due to variations in porosity, pore size, or ceramic material composition.

The bone defect repair system 100 can be assembled and used in a surgical procedure that begins with obtaining computed tomography scans of the patient anatomy. The computed tomography data provides three-dimensional imaging of the bone structure at the defect site, enabling fabrication of patient-specific components including the scaffold segments 110, the sleeve 130, and a surgical cutting guide. The computed tomography data can then be processed using three-dimensional modeling software to generate digital models of each component that conform to the patient-specific bone geometry.

The scaffold segments 110 can be fabricated using the photocasting process described herein, with the scaffold geometry derived from the patient-specific computed tomography data. The sleeve 130 can be fabricated using melt extrusion 3D printing of polycaprolactone, with the interior cavity 132 shaped to conform to the patient-specific bone geometry. A patient-specific surgical cutting guide can be fabricated from a biocompatible surgical guide resin using a stereolithography 3D printer. The surgical guide resin is autoclavable, enabling sterilization of the cutting guide using standard autoclave procedures. In some implementations, the surgical cutting guide is printed as a solid resin structure.

In some implementations, a patient-specific surgical cutting guide can be fabricated from polyethylene terephthalate glycol (PETG) that contacts the distal radius, ulnar head, and metacarpal bones to guide precise osteotomy cuts. In some implementations, the surgical cutting guide is printed with 30% gyroid infill and at least 3 inner perimeters using PETG at 235 °C nozzle temperature.

In some implementations, the surgical cutting guide includes k-wire holes with a diameter of 1.75 mm for temporary fixation to the bone during surgery. In some implementations, the k-wire holes are oriented normal to the surface of the bone to facilitate drilling. In some implementations, the k-wire holes are positioned at opposing angles on either side of the cutting guide, with k-wire holes on one side angled in a first direction and k-wire holes on the opposite side angled in a second direction that mirrors the first direction. When k-wires are inserted through the angled holes on both sides of the cutting guide, the opposing angles create a locking configuration that secures the cutting guide to the bone and prevents the cutting guide from lifting away from the bone surface during the osteotomy procedure.

Prior to implantation, each component of the bone defect repair system 100 is sterilized using sterilization methods appropriate for the component material. In some implementations, the scaffold segments 110 are sterilized by autoclaving at 121 °C and 2 atm for 20 minutes. In some implementations, the sleeve 130 is sterilized by soaking in peracetic acid at 2500 ppm diluted with 20% ethanol followed by rinsing with 0.9% saline solution. In some implementations, the surgical cutting guide is sterilized with an autoclave. In some implementations, the surgical cutting guide is sterilized using low-temperature hydrogen peroxide biodecontamination.

During the surgical procedure, the surgical cutting guide is positioned on the bone anatomy and temporarily secured using k-wires inserted through the k-wire holes in the cutting guide. The bone defect is resected using the cutting guide to direct the osteotomy cuts at the proximal and distal boundaries of the defect region. The cutting guide ensures that the osteotomy cuts are positioned at the locations planned during the preoperative design phase, enabling the patient-specific sleeve 130 and scaffold segments 110 to fit properly within the resulting defect.

After resection of the bone defect, the scaffold segments 110 are prepared for implantation by loading collagen sponges 124 with bioactive agents into the central canals 120 of each scaffold segment 110. The collagen sponges 124 are cut to size and threaded through the central canal 120 of each scaffold segment 110 using suture material. Bone morphogenetic protein is loaded into the collagen sponges 124 by inserting a syringe through the central canal 120 and dispensing the bioactive agent evenly over the length of the collagen sponge 124 within each scaffold segment 110. In some implementations, bioactive agents are applied to an outer surface of the scaffold using a luer lock syringe with a fine needle. Due to the porous architecture of the scaffold, the bioactive agents wick into the interior of the scaffold structure. In some implementations, bioactive agents are applied to an interior surface of the scaffold by inserting a syringe with a long needle through the central canal and dispensing the bioactive agents along the interior walls of the central canal.

The scaffold segments 110 are inserted sequentially into the sleeve 130 with the proximal piece inserted first and the distal piece inserted last. Each scaffold segment 110 is oriented such that the interlocking features 116 engage with the complementary interlocking features 116 on the adjacent scaffold segment 110. The stepped profiles 118 on adjacent scaffold segments 110 mechanically engage as each successive scaffold segment 110 is inserted into the sleeve 130, creating a continuous scaffold structure within the interior cavity 132 of the sleeve 130. The guide rails 142 on each scaffold segment 110 align with the corresponding guide channels in the sleeve 130 to maintain proper rotational alignment of the scaffold segments 110 within the sleeve 130.

Biodegradable plugs 126 are inserted into unused fixation plate screw holes 164 prior to positioning the sleeve assembly. The biodegradable plugs 126 fill the screw holes 164 in the fixation plate 160 that overlie the scaffold region, preventing bone growth through the unused screw holes 164 that would inhibit later removal of the fixation plate 160.

The sleeve 130 containing the assembled scaffold segments 110 is slid onto the fixation plate 160. The channel 134 in the sleeve 130 receives the fixation plate 160 as the sleeve 130 is advanced along the plate. The assembly is positioned between the proximal and distal bone segments 102, 104, with the proximal end 138 of the sleeve 130 overlapping with the proximal host bone segment 102 and the distal end 140 of the sleeve 130 contacting the distal host bone structure.

The fixation plate 160 is secured to the bone with fixation screws 162. Fixation screws 162 are inserted through the screw holes 164 in the fixation plate 160 at the proximal end and driven into the proximal host bone segment 102. Fixation screws 162 are inserted through the screw holes 164 in the fixation plate 160 at the distal end and driven into the distal host bone segment 104. The fixation screws 162 anchor the fixation plate 160 to the native bone tissue, maintaining the position of the entire bone defect repair construct.

The sleeve 130 is secured to the proximal bone with bone fixation screws 148 through the fixation apertures 146 at the proximal end 138 of the sleeve 130. The bone fixation screws 148 extend through the fixation apertures 146 in the sleeve 130 and engage with the cortical bone of the proximal host bone segment 102, providing additional anchoring of the sleeve 130 to the native bone tissue.

Post-operative healing proceeds with the scaffold providing a three-dimensional environment for bone tissue ingrowth while the fixation plate 160 provides mechanical stabilization of the construct. The bone morphogenetic protein loaded into the collagen sponges 124 within the central canals 120 of the scaffold segments 110 promotes osteogenic differentiation and bone formation within the porous scaffold architecture 114. The sleeve 130 protects the brittle ceramic scaffold segments 110 during the healing process while permitting controlled micro-motion that stimulates bone cell activity.

The scaffold segments 110 degrade over time as new bone tissue forms within the porous architecture 114. The hydroxyapatite or beta-tricalcium phosphate ceramic material is resorbed and replaced by native bone tissue through the bone remodeling process. The polycaprolactone sleeve 130 degrades over a timeline of approximately 2 years, with the degradation products absorbed by the body without adverse effects.

After the bone defect has healed and the scaffold has been replaced by native bone tissue, the fixation plate 160 is removed in a subsequent surgical procedure. The biodegradable plugs 126 that filled the unused screw holes 164 in the fixation plate 160 have degraded by the time of plate removal, and the absence of bone growth through the unused screw holes 164 makes removal of the fixation plate 160 easier.

In some implementations, the surgical cutting guide is fabricated from materials other than PETG to accommodate different sterilization requirements or mechanical properties. In some implementations, the k-wire holes in the surgical cutting guide have diameters other than 1.75 mm to accommodate different k-wire sizes. In some implementations, the scaffold segments 110 are sterilized using sterilization methods other than autoclaving, such as gamma irradiation or ethylene oxide sterilization. In some implementations, the sleeve 130 is sterilized using sterilization methods other than peracetic acid, such as ethylene oxide sterilization or low-temperature hydrogen peroxide biodecontamination. In some implementations, the fixation plate 160 is not removed after healing if the patient anatomy or clinical circumstances indicate that plate retention is preferable. In some implementations, the sleeve 130 is not secured to the proximal bone with bone fixation screws 148 if the fixation plate 160 provides sufficient stabilization of the construct.

The sleeve 130 can be printed in more than one piece in some configurations. A multi-piece sleeve configuration accommodates manufacturing constraints associated with large sleeve dimensions or complex geometries that exceed the build volume of available 3D printers. The multi-piece sleeve configuration includes complementary mating features that enable assembly of the sleeve pieces around the scaffold segments 110 and fixation plate 160. The multi-piece sleeve configuration is joined using adhesive bonding, mechanical fasteners, or interference fit between the sleeve pieces.

The system 100 can be used without a fixation plate 160 in less load-bearing applications. In applications where the bone defect is located in a non-weight-bearing region or where the mechanical demands on the repair construct are reduced, the sleeve 130 and scaffold segments 110 can provide sufficient structural support without the additional stabilization provided by a fixation plate 160. The sleeve 130 constrains the scaffold segments 110 within the interior cavity 132 and interfaces with the proximal and distal host bone segments 102, 104 to maintain the position of the repair construct. The fixation apertures 146 in the sleeve 130 enable bone fixation screws 148 to anchor the sleeve 130 directly to the host bone, providing mechanical stabilization in the absence of a fixation plate 160.

Various scaffold topologies are combined within a single bone defect repair system 100. The scaffold segments 110 within a single sleeve 130 can include different triply periodic minimal surface topologies, with some scaffold segments 110 having a gyroid topology and other scaffold segments 110 having a Fischer-Koch S topology. The combination of different topologies within a single repair construct enables optimization of mechanical properties, permeability, and surface area at different locations within the defect region. The scaffold segments 110 with different topologies are configured with compatible interlocking features 116 that enable mechanical engagement between adjacent scaffold segments 110 regardless of the topology of each segment.

Various scaffold materials can be combined within a single bone defect repair system 100. The scaffold segments 110 within a single sleeve 130 include different calcium phosphate-based ceramic materials, with some scaffold segments 110 comprising hydroxyapatite and other scaffold segments 110 comprising beta-tricalcium phosphate. The combination of different materials within a single repair construct enables optimization of degradation rates at different locations within the defect region. Scaffold segments 110 comprising beta-tricalcium phosphate degrade more rapidly than scaffold segments 110 comprising hydroxyapatite, enabling staged resorption of the scaffold structure as bone tissue ingrowth progresses.

The system 100 is applicable to both veterinary and human orthopedic medicine. The bone defect repair system 100 is configured for treatment of critically sized bone defects in canine patients, including defects resulting from osteosarcoma resection in the distal radius. The bone defect repair system 100 is configured for treatment of critically sized bone defects in human patients, including defects resulting from trauma, tumor resection, or bone disease. The patient-specific design approach using computed tomography data enables fabrication of components that conform to the bone geometry of patients across different species and anatomical locations.

The system 100 is also applicable to mandibular defects. The bone defect repair system 100 is configured for treatment of defects in the mandible resulting from tumor resection, trauma, or congenital abnormalities. The scaffold segments 110 and sleeve 130 are designed to conform to the curved geometry of the mandible, with the interior cavity 132 of the sleeve 130 shaped to match the patient-specific mandibular anatomy derived from computed tomography data. The fixation plate 160 is contoured to follow the curvature of the mandible and is secured to the remaining mandibular bone segments using fixation screws 162.

The system 100 is further applicable to segmental defects in long bones other than the radius. The bone defect repair system 100 is configured for treatment of segmental defects in the tibia, femur, humerus, ulna, and metatarsal bones. The scaffold segments 110 and sleeve 130 are designed to conform to the geometry of the affected bone, with the interior cavity 132 of the sleeve 130 shaped to match the patient-specific bone anatomy derived from computed tomography data.

In some implementations, the scaffold can be designed for perfusion at a nominal flow rate of 1 mL/min per scaffold with an average inlet velocity of 0.1 mm/s. The nominal flow rate and inlet velocity are selected to provide physiologically relevant fluid dynamics within the porous scaffold architecture 114 during cell culture. The flow conditions simulate the interstitial fluid flow that occurs within trabecular bone in vivo.

In some implementations, the scaffold is designed to induce wall shear stresses in a range of 0.55-24 mPa for osteogenic differentiation. The wall shear stress range is selected based on published literature indicating that shear stresses within this range promote osteogenic differentiation of mesenchymal stem cells and osteoblast precursor cells. The wall shear stress is determined by the combination of flow rate, scaffold porosity, pore size, and pore geometry.

In some implementations, the sleeve 130 is printed as a single monolithic piece rather than multiple pieces. In some implementations, the system 100 includes a fixation plate 160 in all applications regardless of the load-bearing requirements. In some implementations, all scaffold segments 110 within a single sleeve 130 have the same triply periodic minimal surface topology. In some implementations, all scaffold segments 110 within a single sleeve 130 comprise the same calcium phosphate-based ceramic material. In some implementations, the system 100 is configured for treatment of bone defects in species other than canine or human patients, including equine, feline, and ovine patients. In some implementations, the perfusion bioreactor has fewer than four channels or more than four channels to accommodate different experimental designs. In some implementations, the elastomeric sleeve has a Shore hardness other than 50 A to accommodate different scaffold surface textures or sealing requirements. In some implementations, the bioreactor body is printed at resolutions other than 100 microns to accommodate different dimensional accuracy requirements. In some implementations, the nominal flow rate is outside the range of 0.5-5 mL/min per scaffold to achieve different shear stress conditions. In some implementations, the wall shear stress is outside the range of 0.55-24 mPa to study cell behavior under different mechanical stimulation conditions.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. A modular bone defect repair system, comprising:
a plurality of scaffold segments, each scaffold segment having a porous architecture configured for bone tissue ingrowth, wherein each scaffold segment includes at least one interlocking feature comprising a stepped profile configured to mechanically engage with a complementary stepped profile on an adjacent scaffold segment; and
a sleeve having an interior cavity configured to receive the plurality of scaffold segments in an assembled configuration, wherein the sleeve constrains relative motion between the assembled scaffold segments.

2. The modular bone defect repair system of claim 1, wherein the sleeve further immobilizes the scaffold segments relative to the host bone.

3. The modular bone defect repair system of claim 1, wherein the porous architecture comprises a triply periodic minimal surface structure.

4. The modular bone defect repair system of claim 3, wherein the triply periodic minimal surface structure is selected from the group consisting of a gyroid topology and a Fischer-Koch S topology.

5. (canceled)

6. The modular bone defect repair system of claim 1, wherein each scaffold segment comprises a calcium phosphate-based ceramic material.

7. The modular bone defect repair system of claim 6, wherein the calcium phosphate-based ceramic material is selected from the group consisting of hydroxyapatite, beta-tricalcium phosphate, and a biologically-derived bone powder.

8. (canceled)

9. (canceled)

10. The modular bone defect repair system of claim 1, wherein each scaffold segment has a porosity in a range of 50% to 90%.

11. (canceled)

12. The modular bone defect repair system of claim 1, wherein each scaffold segment includes a central canal extending therethrough configured to receive a growth factor delivery medium.

13. (canceled)

14. (canceled)

15. The modular bone defect repair system of claim 1, wherein the sleeve comprises a biodegradable polymer.

16. The modular bone defect repair system of claim 15, wherein the biodegradable polymer comprises polycaprolactone.

17. The modular bone defect repair system of claim 1, wherein the sleeve includes a channel extending along a longitudinal axis of the sleeve and configured to receive a fixation plate.

18. (canceled)

19. The modular bone defect repair system of claim 17, further comprising one or more biodegradable plugs configured to fill unused screw holes in the fixation plate, wherein the biodegradable plugs are configured to prevent bone growth through the unused screw holes.

20. (canceled)

21. The modular bone defect repair system of claim 1, wherein the sleeve includes a plurality of fenestrations configured to permit transcortical fluid flow.

22. The modular bone defect repair system of claim 1, wherein the sleeve includes at least one fixation aperture configured to receive a bone fixation screw.

23. The modular bone defect repair system of claim 1, wherein the stepped profile comprises a stair-step configuration having a step height of at least 5 mm.

24. (canceled)

25. The modular bone defect repair system of claim 1, wherein each scaffold segment includes a guide rail configured to align with a corresponding guide channel in the sleeve.

26. The modular bone defect repair system of claim 1, wherein the sleeve has a proximal end configured to contact with a proximal host bone and a distal end configured to contact a distal host bone, wherein the proximal end is configured to overlap with a proximal host bone.

27. (canceled)

28. The modular bone defect repair system of claim 26, wherein the sleeve has a tapered configuration along its longitudinal axis to constrain axial motion of the assembled scaffold segments.

29. The modular bone defect repair system of claim 1, wherein the interior cavity of the sleeve is shaped to conform to a patient-specific bone geometry derived from computed tomography data, wherein the interior cavity of the sleeve conforms to the patient-specific bone geometry to minimize rotational movement of the assembled scaffold segments relative to the host bone.

30. (canceled)

31. The modular bone defect repair system of claim 1, wherein each scaffold segment has a pore size in a range of 300 micrometers to 1200 micrometers.

32.-59. (canceled)
